(19) United States
(12) Patent Application Publication
(10) Pub. No.: US 2005/0256527 A1
(43) Pub. Date: Nov. 17, 2005

(54) Ligament tensioning device with cutting jig, and osteotomy method

(76) Inventors: Daniel Delfosse, Bern (CH); Walter Supper, Bettlach (CH); Beat Grunder, Worb (CH)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/509,989

(22) PCT Filed: Feb. 26, 2003
(86) PCT No.: PCT/EP03/01973

§ 371 Date: May 23, 2005

(30) Foreign Application Priority Data
Apr. 8, 2002 (DE) 102 15 358.2

(51) Int. Cl. A61F 2/00

(52) U.S. Cl. 606/88

(57) ABSTRACT

A ligament-tensioning device for preparing for the implantation of a joint implant has a base body, having a first claw with a distal bearing surface which rests on a first bone, and a second claw which rests, with a proximal bearing surface, against a second bone. The second claw is displaceable parallel to the first claw. A cutting jig can be placed onto mounts of the base body of the ligament-tensioning device.

The invention relates to a ligament-tensioning device with a cutting jig for joints of the human or animal body, and to a procedure for the osteotomy of these joints using the ligament-tensioning device with cutting jig according to the invention.

A ligament-tensioning device for non-spherical joints is known from WO 00/78225 A1. The device described therein for tensioning ligaments in non-spherical joints in the human or animal body comprises a prismatic, cylindrical or plate-shaped base body with a right claw and a left claw, which have first bearing surfaces in one plane and can thus be brought to bear in parallel on the joint-side surface of a first bone adjoining a non-spherical joint, and also a right handle and a left handle, a right tensioning lever and a left tensioning lever with two bearing surfaces arranged parallel to the first bearing surfaces, it being possible to set a span Y between the respective bearing surfaces of the right tensioning lever and the right claw and the same or another span X between the respective bearing surfaces of the left tensioning lever and the left claw. The second bearing surfaces can be brought to bear on the joint-side surface of a second bone adjoining the joint. Furthermore, the device comprises a right operating lever and a left operating lever, which, at the same time as the device is being held by one hand each on the corresponding handle, can be actuated individually by the same hand in each case, and a right parallel-displacement device and a left parallel-displacement device, which can each be driven by the corresponding operating lever and are connected to one tensioning lever each so that when the operating levers are moved the respective spans X and Y can be set independently of one another. The parallel-displacement devices are designed as four-bar-linkage lever mechanisms.

The disadvantage of the ligament-tensioning device known from WO 00/78225 A1 is, in particular, that the provision of cut planes in a diseased joint for the purpose of introducing a prosthesis requires further tools, which are attached to the joint independently of the tensioning device and as a result do not allow precise positioning and alignment, and reproducible, precise cutting.

Accordingly, the object on which the invention is based is to provide a ligament-tensioning device and a procedure for tensioning with a parallel spreading movement the capsule-ligament structures of a joint to be provided with a prosthesis and at the same time enabling presettable, adjustable and checkable cutting when preparing for and carrying out the cuts required for providing a joint with a prosthesis.

The object is achieved with regard to the ligament-tensioning device by the features of claim 1 and with regard to the procedure by the features of claim 20.

Further advantageous refinements of the invention are characterised in the subclaims.

Advantageously, the cutting jig has projections with U-shaped slots which can be fitted into the mounts of the ligament-tensioning device and fixed in a catching manner by means of a locking element.

It is also advantageous that, by way of two corresponding scales which define the particular position of the ligament-tensioning device, it is possible to preset the width of the knee joint gap and the thickness of the implant to be introduced and check it at any time during the operation.

The cutting jig advantageously has a saw guide which enables guidance of the bone saw with very few misalignments and a high degree of cutting accuracy. Furthermore, a cylindrical guide which enables further operating instruments to be attached to the cutting jig is preferably provided.

In a further advantageous embodiment, the ligament-tensioning device can also be designed as a bilateral ligament-tensioning device which enables simultaneous provision for the medial and lateral joint part.

The invention is explained in more detail below with the aid of, in part, schematic illustrations of the preparation for providing a human knee joint with a prosthesis.

In the illustrations:

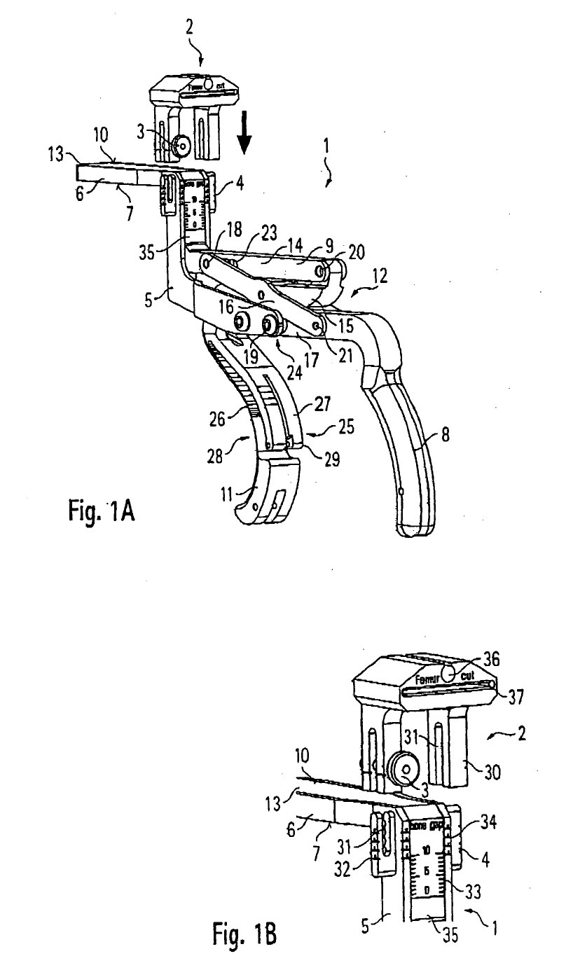

FIG. 1A shows a schematic, perspective view of a ligament-tensioning device with a cutting jig constructed in accordance with the invention,

FIG. 1B shows an enlarged illustration of the cutting jig illustrated in FIG. 1A,

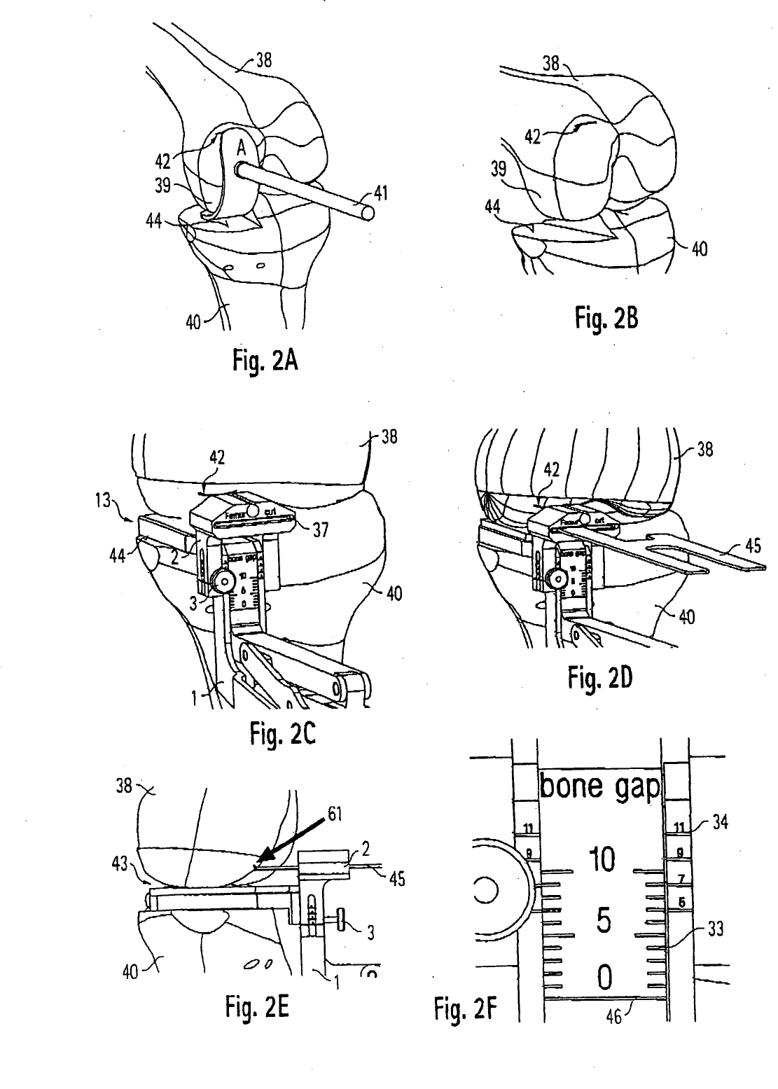

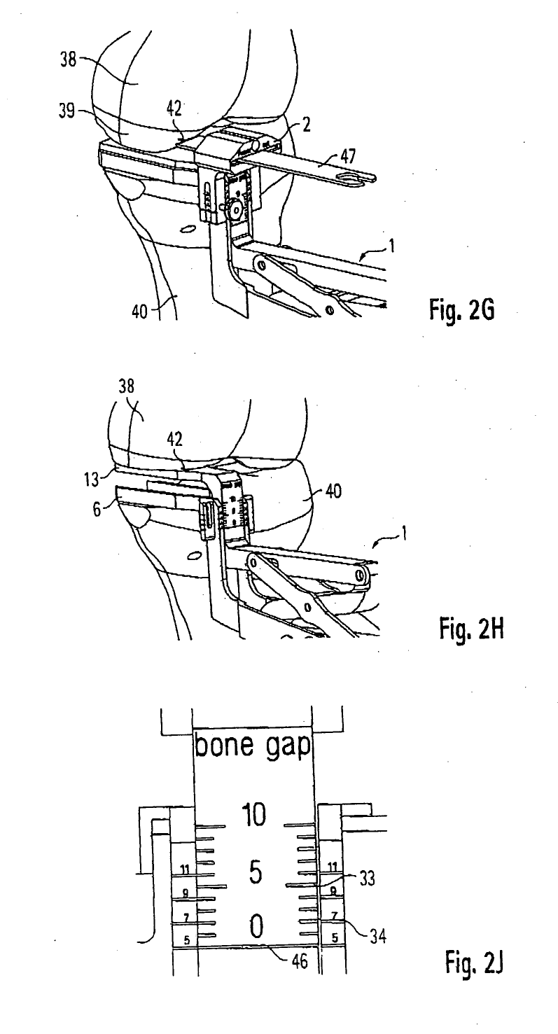

FIGS. 2A-J show schematic, perspective illustrations of a distal femur osteotomy using the cutting jig according to the invention,

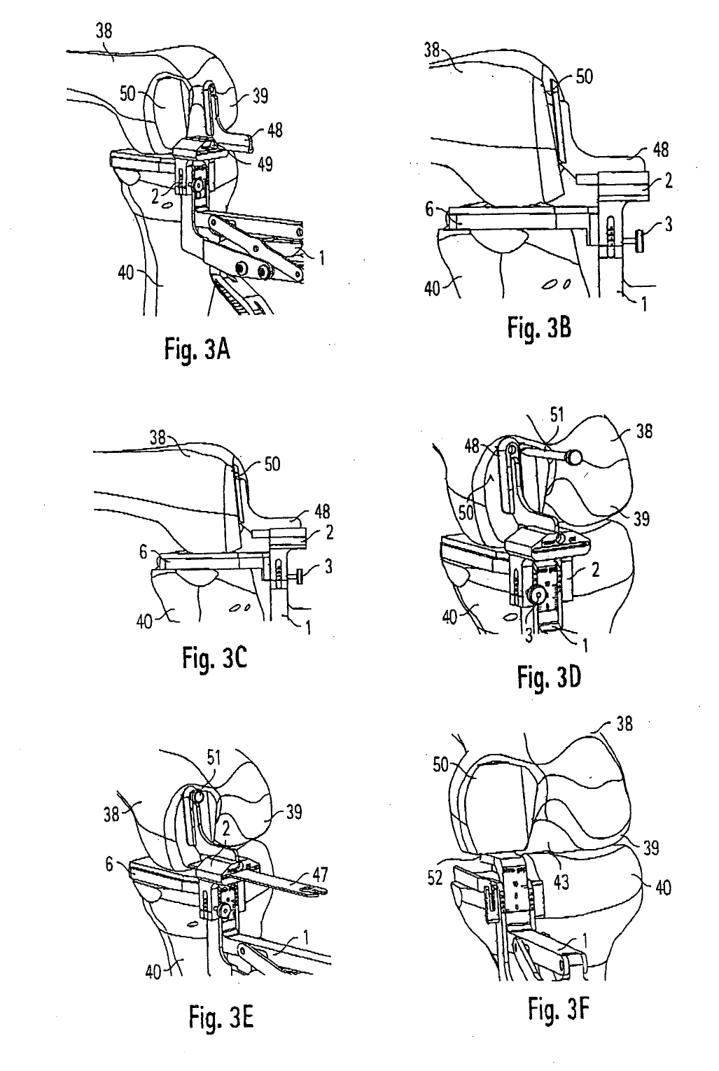

FIGS. 3A-F show schematic, perspective illustrations of a dorsal femur osteotomy using the cutting jig according to the invention, and

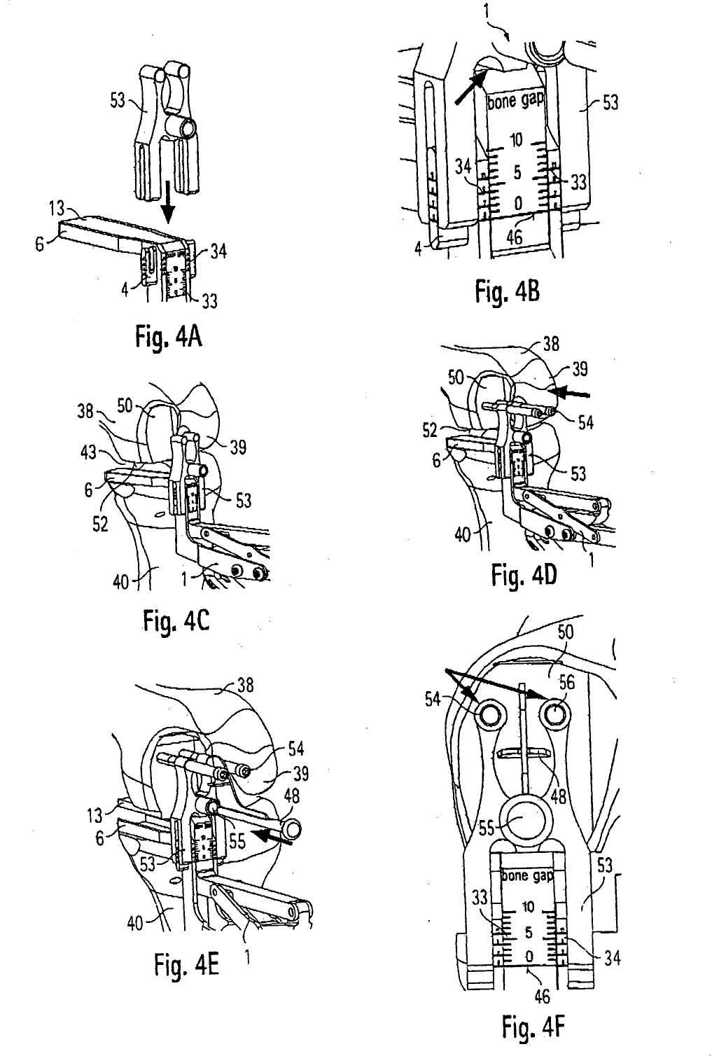

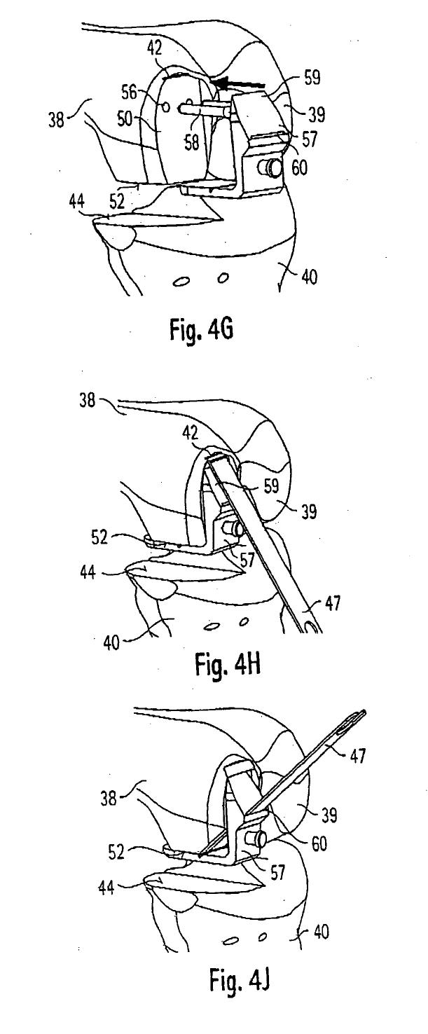

FIGS. 4A-J show schematic, perspective illustrations of femoral oblique cuts using the cutting jig according to the invention.

FIG. 1A shows, in a schematic, perspective general illustration, a ligament-tensioning device 1, onto which a cutting jig 2 can be fitted. The cutting jig 2 can be fitted onto mounts 4 of the ligament-tensioning device 1 and locked to these mounts by means of a locking device 3.

The ligament-tensioning device 1 comprises a base body 5, which, for reliably transmitting the spreading force to the tibia, has a first claw 6 with a bearing surface 7 which is distal in relation to the knee joint gap and rests on the femur in the case of a knee joint. Lying opposite the first claw 6, a handle 8 is correspondingly attached to the base body 5 and this handle permits one-handed holding and tensioning of the ligament-tensioning device 1. Likewise corresponding to the arrangement of the first claw 6 and lying thereabove, the ligament-tensioning device 1 comprises a tensioning lever 9 which is supported, by its proximal bearing surface 10 formed on a second claw 13, on the opposite portion of the joint to be treated, the tibia in the case of a knee joint. The spreading action is produced by actuating the handle 8 together with an operating lever 11 in each case either for a medial or lateral joint portion.

A parallel-displacement device 12 permits, with respect to the bearing surfaces 7 and 10, a parallel displacement of the second claw 13 with the bearing surface 10 relative to the first claw 6 with the bearing surface 7. The second claw 13 is operatively connected to the tensioning lever 9 in this device. The parallel-displacement device 12 is designed as a four-bar linkage in the form of intersecting bars and comprises four levers 14, 15, 16, 17, a lever 14 on the tensioning lever and a lever 17 on the base body being arranged in parallel, while the levers 15 and 16 intersect. The four levers 14, 15, 16, 17 are connected to one another by means of five pivots 18, 19, 20, 21, 22. Two of the pivots 18, 19 are displaceably mounted in the parallel levers 14, 17 in elongated holes 23, 24 running parallel to the bearing surfaces 7, 10. This construction of the parallel-displacement device 12 permits the lever 14 on the tensioning lever and the lever 17 on the base body to be movable parallel to one another or apart from one another. The lengths of the levers 14, 15, 16, 17 are chosen such that, for any span X between the bearing surface 7 on the first claw 6 and the bearing surface 10 on the second claw 13, which may be for example between 5 mm and 40 mm, there is a constant conversion ratio of 1:1 between the tensioning force applied manually to the handle 8 and to the operating lever 11 and the distraction force exerted on the bones adjoining the joint.

The size of the spreading force can be read off on a force indicator 25 with a scale 26 and a movable indicating lever 27. The indicating lever 27 is moved by the longitudinal bending of the operating lever part 28, which is bendable by a manually applied tensioning force, relative to the other indicating lever 27, which is arranged in the manner of a fork and is not acted upon by this tensioning force. If the indicating lever 27 and the operating lever part 28 are moved relative to one another by means of the tensioning force, the indicating lever 27 rotates about an axis of rotation 29, whereby the manually applied tensioning force is indicated on the scale 26 by the indicating lever 27.

Furthermore, provision may be made between the handle 8 and the operating lever 11 for a locking device, not illustrated specifically in FIG. 1A, which enables the ligament-tensioning device 1 to be locked in a particular position.

In the exemplary embodiment, the first claw 6 and the second claw 13 are formed as an outer claw 6 and an inner claw 13, which, in the relaxed state of the ligament-tensioning device 1, lie in one plane and form a continuous distal and proximal bearing surface. This facilitates the introduction of the ligament-tensioning device 1 into the joint to be treated. On actuation of the ligament-tensioning device 1, the inner claw 13 is parallel-displaced relative to the outer claw 6.

The base body 5 of the ligament-tensioning device 1 has a first scale 34, which corresponds with a second scale 33 on a component 35 connecting the lever 14 to the second claw 13. The scales 33 and 34 serve for presetting and checking the width of the knee joint gap before and after the osteotomies preparing for the implantation. The precise function of the scales 33 and 34 is explained in more detail in FIG. 2F and in the associated description.

The ligament-tensioning device 1 may also be designed as a bilateral ligament-tensioning device 1 with two ligament-tensioning devices 1 which act parallel to one another, can be connected to one another in any desired way, for example in the region of the base body 5, and enable simultaneous provision for the medial and the lateral joint portion. In this case, one or two cutting jigs 2 capable of being placed onto the ligament-tensioning device 1 are then to be provided as well.

FIG. 1B shows, in an enlarged view, the region of the ligament-tensioning device 1 in which the cutting jig 2 is mounted. The cutting jig 2 has two projections 30 of U-shaped design, forming slots 31 which come into engagement with the mounts 4 on the base body 5 of the ligament-tensioning device 1 during the fitting of the cutting jig 2. One of the mounts 4 is likewise of U-shaped design, the slot 31 thereby formed having catches 32, which come into engagement with the locking element 3 so that the cutting jig 2 is displaceably lockable on the base body 5 of the ligament-tensioning device 1 in, for example, equidistant steps of, for example, 2 mm.

The cutting jig 2 furthermore has a cylindrical guide 36, into which further instruments for carrying out or positioning the osteotomies can be introduced. This may, for example, as can be seen in FIGS. 3A to 3E, be an aligning jig 48, which enables fixed positioning of the cutting jig 2 at a specified angle.

For guidance of a feeler gauge or the bone saw to be used for the osteotomies, provision is made for a saw guide 37 which is preferably formed at right angles in the cutting jig 2. The saw guide 37 ensures guidance of the saw in a manner free from deviation, whereby a high degree of accuracy with few misalignments is achieved during the resection of the bone parts concerned.

The following FIGS. 2A to 2J to 4A to 4J show the working steps required to prepare the femur 38 in the region of the knee joint for the implantation of an implant which replaces a femur condyle 39 destroyed, for example, by arthrosis. The preparatory measures on the tibia 40 can be carried out by means of resection methods which are already known.

FIGS. 2A to 2J show the preparatory work for the distal femur osteotomy. For this purpose, firstly, as not explained in any more detail, the tibia 40 is appropriately prepared, as can already be seen in FIG. 2A. It can also be seen from FIG. 2A how the size of the femur 39 to be resected is determined by means of a femur size gauge 41. Several sizes, e.g. five, for correctly determining the size of the femur 38 are available to the surgeon for this. Of interest for the distal femur osteotomy is firstly the mark, indicated by 42 in FIG. 2A, which is made on the femur 38. For this, the leg is firstly put into a 90° position and, after positioning the femur size gauge 41 on the ventral end, a mark 42 is made on the femur 38 with the aid of a cautery knife. The position of this mark is roughly the boundary between the femorotibial and the femoropatellar part of the femur 38. After removing the femur size gauge 41, the mark 42 made is visible on the femur 38, as can be seen in FIG. 2B.

In the next step, as illustrated in FIG. 2C, the cutting jig 2 is premounted on the ligament-tensioning device 1, the cutting jig 2 being fitted onto the ligament-tensioning device 1, as described in FIGS. 1A and 1B. With the aid of the locking element 3, the thickness of the inlay implant to be inserted later can be set at between 5 and 11 mm in 2 mm steps, as already mentioned above.

After that, with the leg extended, the ligament-tensioning device 1 with the cutting jig 2 mounted is introduced into the joint gap 43. In so doing, the distal bearing surface 7 of the first claw 6 of the ligament-tensioning device 1 rests on the cut tibia surface 44 which has already been prepared.

Now, the ligament-tensioning device 1 is spread with the desired force. A feeler gauge 45 is passed through the saw guide 37 of the cutting jig 2 until the feeler gauge 45 rests against the femur 38. Now, it is checked whether the mark 42 on the femur 38 is in line with the feeler gauge 45. This can be seen from FIGS. 2D and 2E. The arrow 61 in FIG. 2E shows clearly that the feeler gauge 45 is now resting in the desired position against the mark 42 and that the cutting path for the distal femur osteotomy is correctly set. This can be optimally set by a specific movement of the lower leg.

After the spreading of the ligament-tensioning device 1, the thickness of the distal femur condyle 39 to be resected can be read off using the gradation on the scale 34 of the ligament-tensioning device 1 before the actual resection. The distance is determined from the difference between the zero line 46 and the chosen thickness of the implant to be inserted later. In FIG. 2F, the condyle thickness to be resected is 7 mm if a 5 mm inlay implant is selected. If the measured value is less than 5 mm, the cutting jig 2 has to be set accordingly for a greater implant thickness, e.g. for 7 or 9 mm. If the measured difference is greater than 8 mm, e.g. for a set condyle height of 5 mm, a further resection of the tibia 40 must be performed.

After the preparatory work, the distal femur osteotomy is carried out by means of a saw 47 passed through the saw guide 37 of the cutting jig 2. This is illustrated in FIG. 2G.

After carrying out the distal femur osteotomy, the ligament-tensioning device 1 is relaxed and removed from the knee joint gap 43. The cutting jig 2 is demounted from the ligament-tensioning device 1. After that, the ligament-tensioning device 1 is again introduced into the joint gap 43. Now, the width of the joint gap 43 is checked by spreading the ligament-tensioning device 1. The zero line 46 must correspond to the chosen implant thickness, as illustrated in FIG. 2H and FIG. 2J.

In the example, the correspondence with the preselected implant thickness of 5 mm can be seen, since the zero line 46 of the scale 33 now coincides with the 5 mm line of the scale 34, as illustrated in FIG. 2J. In the event of deviations of more than one millimetre, a correction must be made by further resection of the tibia 40 or of the distal femur condyle.

FIGS. 3A to 3F show the step of the dorsal femur osteotomy which now follows. For this purpose, the leg is firstly put into a 90° flexed position again.

The cutting jig 2 is again premounted on the ligament-tensioning device 1. In so doing, the cutting jig 2 is set to the same inlay implant thickness as in the preceding distal femur osteotomy. The premounted ligament-tensioning device 1 is now introduced into the joint gap 43. After that, the ligament-tensioning device 1 is spread with the desired force.

Now, as illustrated in FIG. 3A, an aligning jig 48 for the dorsal femur cut is pushed into a cylindrical guide 49 of the cutting jig 2. The aligning jig 48 is now displaced until it is in contact with the distal femur surface 50, as illustrated in FIG. 3B. Now, as shown in FIG. 3C, by moving the lower leg, the position of the aligning jig 48 has to be set so that the latter rests evenly against the distal femur surface 50.

After checking the spreading force, the aligning gauge 48 can be fixed to the distal femur surface 50 using a bone nail 51, as illustrated in FIG. 3D. The system is thereby stabilised. It must be ensured here that the aligning jig 48 is still resting evenly against the distal femur surface 50.

After that, the saw 47 is introduced through the saw guide 37 of the cutting jig 2 as in the distal femur osteotomy, and the dorsal femur osteotomy is carried out. Care must be taken here to protect the ligamentary structures.

After carrying out the dorsal femur osteotomy, the bone nail 51 and the ligament-tensioning device 1 are removed. After that, the cutting jig 2 is removed from the ligament-tensioning device 1. Where necessary, dorsal osteophytes are removed by subsequent treatment.

After that, as illustrated in FIG. 3F, the ligament-tensioning device 1 is once again introduced into the joint gap 43. The flexion gap is checked using the marks on the scale, as already described in FIGS. 2A to 2J. If the desired flexion gap does not correspond to the measured gap, it is necessary to carry out further resection of the dorsal femur surface 52 (repetition of the steps illustrated in FIGS. 3A to 3F).

FIGS. 4A to 4J illustrate the working steps for the concluding femoral oblique cuts. In this case, firstly, as can be seen from FIG. 4A, a drilling jig 53, required to drill holes for the attachment of an oblique-cutting block, is mounted on the ligament-tensioning device 1. The drilling jig 53 is pushed into the mounts 4, which are also used for the cutting jig 2. The drilling jig 53 must be mounted as far as it will go on the ligament-tensioning device 1, as can be seen from FIG. 4B.

After that, the ligament-tensioning device 1 is introduced into the knee joint gap 43 as in the previous steps, as can be seen from FIG. 4C. After that, two drilling sleeves 54 are pushed in through the drilling-jig 53 until they butt against the distal femur surface 52. This is illustrated in FIG. 4D. Furthermore, as can be seen from FIG. 4E, the ligament-tensioning device 1 is spread with the desired force. It is again checked whether the drilling jig 53 is resting flat on the ligament-tensioning device 1. Now, the aligning jig 48, which has already been used in the dorsal femur osteotomy, is pushed into the cylindrical guide 55, as illustrated in FIG. 4E, and the position of the aligning jig 48 is again set so that the aligning jig 48 rests evenly against the distal femur surface 50. When the tensioner is set precisely, the zero mark 46 should correspond to the desired inlay thickness 34.

After a further check of the optimal tension of the ligament-tensioning device 1, two holes 56 with a drilling depth of about 35 mm and a diameter of about 3.2 mm are drilled into the distal femur surface 50, according to FIG. 4F. After that, the ligament-tensioning device 1 is loosened and removed from the knee joint gap 43.

FIG. 4G shows the oblique-cutting jig 57, suitably selected in each case, being pushed into the two holes 56 by way of two corresponding pins 58. The oblique-cutting jig 57 can be pushed in, or else driven in with guidance by means of holding pincers. The oblique-cutting jig 57 is designed so that it rests straight against the distal femur surface 50 and the dorsal femur surface 52.

The oblique-cutting jig 57 has a bearing surface 59 for making the first, ventral oblique cut and a saw guide 60 for the second, dorsal oblique cut.

FIG. 4H shows the ventral oblique cut, which is made up to the ventral mark 42, which has been made on the femur 38 at the start of the operation using a cautery knife.

FIG. 4J shows the concluding dorsal oblique cut, the saw 47 being passed through the saw guide 60.

After removal of the oblique-cutting jig 57, finishing work is carried out on the tibia 40 and the femur 38, and finally the femoral and tibial implants are implanted.

The invention is not restricted to the exemplary embodiment illustrated and—as already mentioned—can also be employed for bilateral implants in the knee joint. The basic principle of providing mounts for a cutting jig on a suitably adapted ligament-tensioning device can also be applied to other joints.

1. Ligament-tensioning device for preparing for the implantation of a joint implant, with a base body, having a first claw with a distal bearing surface which rests on a first bone, and a second claw which rests, with a proximal bearing surface, against a second bone, the second claw being displaceable parallel to the first claw, characterised in that wherein a cutting jig can be placed onto mounts of the base body of the ligament-tensioning device.

2. Ligament-tensioning device according to claim 1, wherein the cutting jig has U-shaped projections with slots.

3. Ligament-tensioning device according to claim 2, wherein the projections of the cutting jig can be brought into engagement with the mounts.

4. Ligament-tensioning device according to claim 1, wherein the cutting jig can be is fixed to the mounts by means of a locking element.

5. Ligament-tensioning device according to claim 1, wherein the mounts comprise catches.

6. Ligament-tensioning device according to claim 5, wherein the catches are equidistant.

7. Ligament-tensioning device according to claim 5 wherein the cutting jig is displaceable on the mounts in a catching manner.

8. Ligament-tensioning device according to claim 1, wherein the first claw and the second claw are displaceable parallel to one another by means of a parallel-displacement device.

9. Ligament-tensioning device according to claim 8, wherein a first scale is provided on a component connecting the second claw to the parallel-displacement device.

10. Ligament-tensioning device according to claim 8, wherein a second scale is provided on the base body.

11. Ligament-tensioning device according to claim 10, wherein the first and second scales can be brought into coincidence so that the height of an implant to be inserted into the joint to be treated can be preset.

12. Ligament-tensioning device according to claim 1, wherein the cutting jig has a cylindrical guide.

13. Ligament-tensioning device according to claim 12, wherein an aligning jig can be introduced into the cylindrical guide.

14. Ligament-tensioning device according to claim 13, wherein the aligning jig can be fixed to the second bone by means of a bone nail.

15. Ligament-tensioning device according to claim 1, wherein the cutting jig has a saw guide.

16. Ligament-tensioning device according to claim 1, wherein a drilling jig can be fitted onto the ligament-tensioning device.

17. Ligament-tensioning device according to claim 16, wherein the drilling jig can be placed onto the mounts of the base body.

18. Ligament-tensioning device according to claim 1, wherein the ligament-tensioning device is a bilateral ligament-tensioning device.

19. Ligament-tensioning device according to claim 18, wherein the ligament-tensioning device has a force indicator.

20. Procedure for preparing a joint for the implantation of a joint implant by means of a ligament-tensioning device with cutting jig, the ligament-tensioning device comprising a base body, having a first claw with a distal bearing surface which rests on a first bone, and a second claw which rests, with a proximal bearing surface, against a second bone, the second claw being displaceable parallel to the first claw, and the cutting jig being able to be placed onto mounts of the base body of the ligament-tensioning device, the procedure comprising the steps of:
carrying out a distal femur osteotomy while simultaneously tensioning the ligaments by means of the ligament-tensioning device,
carrying out a dorsal femur osteotomy while simultaneously tensioning the ligaments by means of the ligament-tensioning device, and
carrying out femoral oblique cuts while simultaneously tensioning the ligaments by means of the ligament-tensioning device.

21. Procedure according to claim 20, wherein the joint implant is a knee joint implant which is implanted into the tibia and the femur.

22. Procedure according to claim 20, wherein the first procedure step comprises the substeps of:
premounting the cutting jig on the ligament-tensioning device,
setting the desired thickness of the implant,
introducing the ligament-tensioning device into the knee joint gap,
spreading the ligament-tensioning device with a predetermined force,
introducing a feeler gauge into a saw guide of the cutting jig,
checking the distal femur cutting path,
carrying out the distal femur osteotomy by means of a saw passed through the saw guide of the cutting jig,
removing the ligament-tensioning device from the knee joint gap,
demounting the cutting jig,
reintroducing the ligament-tensioning device into the knee joint gap, and
checking the width of the knee joint gap by means of scales s present on the ligament-tensioning device.

23. Procedure according to claim 20, wherein the second procedure step comprises the substeps of:
flexing the leg,
premounting the cutting jig on the ligament-tensioning device,
introducing the ligament-tensioning device into the knee joint gap,
spreading the ligament tensioning device with a predetermined force,
pushing the aligning jig for the dorsal femur cut into a cylindrical guide of the cutting jig,
displacing the aligning jig up against the distal femur surface,
adjusting the lower leg until the aligning jig rests evenly against the distal femur surface,
fixing the aligning jig to the distal femur surface by means of a bone nail,
dorsal femur osteotomy,
removing the bone nail,
removing the ligament-tensioning device from the knee joint gap,
demounting the cutting jig,
removing the dorsal osteophytes,
reintroducing the ligament-tensioning device into the knee joint gap, and
checking the width of the knee joint gap by means of scales present on the ligament-tensioning device.

24. Procedure according to claim 20, wherein the third procedure step comprises the following substeps of:
mounting a drilling jig for an oblique-cutting jig as far as it will go on the ligament-tensioning device,
introducing the ligament-tensioning device into the knee joint gap,
pushing two drilling sleeves through the drilling jig up to the distal femur surface,
spreading the ligament-tensioning device with a predetermined force,
pushing the aligning jig for the dorsal femur cut into a cylindrical guide of the drilling jig,
displacing the aligning jig up against the distal femur surface,
adjusting the lower leg until the aligning jig rests evenly against the distal femur surface,
drilling two holes in the distal femur surface,
removing the ligament-tensioning device from the knee joint gap,
pushing the chosen oblique-cutting jig into the two holes,
resecting the ventral oblique cut up to the mark, and
resecting the dorsal oblique cut.
